(19) United States
(12) Patent Application Publication
(10) Pub. No.: US 2013/0152947 A1
(43) Pub. Date: Jun. 20, 2013

(54) SLEEP MASK

(71) Applicant: Rudolf H. Binder, Tucson, AZ (US)

(72) Inventor: Rudolf H. Binder, Tucson, AZ (US)

(21) Appl. No.: 13/709,659

(22) Filed: Dec. 10, 2012

Related U.S. Application Data
(60) Provisional application No. 61/576,481, filed on Dec. 16, 2011.

(51) Int. Cl. A61F 9/04

(52) U.S. Cl. CPC A61F 9/04
USPC 128/858

(57) ABSTRACT

A system and method for restricting a movement of a head of a person wearing a sleep mask. A system includes a non-stretchable harness attached or attachable to the sleep mask and affixable to an object distant to the person's head such as to impede the head's movement in at least one of forward and shoulder-to-shoulder direction.

### CROSS-REFERENCE TO RELATED APPLICATIONS

The present patent application claims priority from and benefit of the U.S. Provisional Patent Application No. 61/576,481 filed on Dec. 16, 2011 and titled “Sleep Mask System and Method,” the disclosure of which is incorporated herein in its entirety for all purposes.

### TECHNICAL FIELD

The present invention relates to sleep masks and, in particularly, to a sleep mask adapted to facilitate maintaining a position of a head of a person wearing the mask while sitting or standing.

### SUMMARY OF THE INVENTION

An embodiment of the present invention provides a sleep mask system that includes a mask portion and a suspending portion. The mask portion contains a blindfolding component configured to cover eyes of the user such as to block ambient light from reaching the eyes, and a substantially non-stretchable harness affixed to the blindfolding component such as to circumscribe the head of the user and to fasten the blindfolding portion over the eyes of the user. The suspending portion is substantially non-stretchable and has a proximal end attached or attachable to the harness at a point opposite the blindfolding portion and a distal end equipped with affixing means. The affixing means are configured to ensure that the suspending portion can be tensionably cooperated with an element of clothing of the user such as to restrict the movement of the head of the user, who wears the sleep mask system, in a forward direction. The sleep mask system can additionally include a substantially non-stretchable side-stabilizing portion having an end attached to the first harness in proximity to the blindfolding portion and adapted to restrict a side movement of the user's head. Either of the suspending and side-stabilizing portions optionally has an adjustable fit defined with respect to the user wearing the system.

Another embodiment provides a method for stabilizing a position of a head of a person. Such method includes providing a sleep mask that contains a blindfolding portion configured to cover eyes of the person such as to block ambient light from reaching the eyes. The sleep mask can further contain a substantially non-stretchable harness affixed to the blindfolding portion such as to circumscribe the head of the user and to fasten the blindfolding portion over the eyes of the user. The method further includes providing a substantially non-stretchable portion having a first end attached or attachable to the first harness and a second end removably affixable to an element of clothing of the person such as to restrict a movement of the person's head in at least one of forward and sideway directions.

### BRIEF DESCRIPTION OF THE DRAWINGS

The invention will be more fully understood by referring to the following Detailed Description in conjunction with the Drawings, of which:

FIG. 1 is a side view is a sitting person who is relaxed and asleep, with the person's head fallen down forward towards her chest.

FIGS. 2A, 2B, and 2C are schematic illustration of components and/or embodiments of a sleep mask system of the present invention.

FIGS. 3A, 3B, 3C are schematic diagrams showing a vertical-strap component of an embodiment of FIG. 2C removably attached to an element of person's clothing with affixing means.

FIGS. 4A and 4B are perspective views of examples of affixing means for use with an embodiment of the invention.

FIG. 4C is perspective view of the embodiment of FIG. 2C.

FIGS. 5, 6, and 7 are schematic views of alternative embodiments of the invention.

FIG. 8 is a diagram of yet another alternative embodiment of the invention.

FIGS. 9A, 9B, 9C depict a person wearing an embodiment of the sleep mask system while seating or standing.

### DETAILED DESCRIPTION

References throughout this specification to “one embodiment,” “an embodiment,” “a related embodiment,” or similar language mean that a particular feature, structure, or characteristic described in connection with the referred to “embodiment” is included in at least one embodiment of the present invention. Thus, appearances of the phrases “in one embodiment,” “in an embodiment,” and similar language throughout this specification may, but do not necessarily, refer to the same implementation of the invention. It is to be understood that no portion of disclosure, taken on its own and in possible connection with a figure, is intended to provide a complete description of all features of the invention.

In addition, the following disclosure may describe features of the invention with reference to corresponding drawings, in which like numbers represent the same or similar elements wherever possible. In the drawings, the depicted structural elements are generally not to scale, and certain components are enlarged relative to the other components for purposes of emphasis and understanding. It is to be understood that no single drawing is intended to support a complete description of all features of the invention. In other words, a given drawing is generally descriptive of only some, and generally not all, features of the invention. A given drawing and an associated portion of the disclosure containing a description referencing such drawing may not, generally, contain all elements of a particular view or all features that can be presented is this view. This is done for the purposes of simplifying a given drawing and the discussion of it, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed.

Accordingly, the invention as recited in claims appended to this disclosure is intended to be assessed in light of the disclosure as a whole.

Various incarnations of so-called “sleep mask” are known in related art. A typical sleep mask (sometimes also referred to as “sleep eye mask” or “eye mask”) is a personal care product and includes, in relevant part, an optionally-layered piece of cloth or other flexible garment that is appropriately shaped for use as a blindfold, and that is often tied to one's head with a stretchable strap to cover the eyes to disable the wearer's sight. Such mask blocks out light when sleeping during the day or, for example, during air travel. A sleep mask is also known to provide relief from claustrophobia. While most sleep masks are adapted to only cover the wearer's eyes, some of the masks are extended across the face such as to include an opening for the wearer's nose.

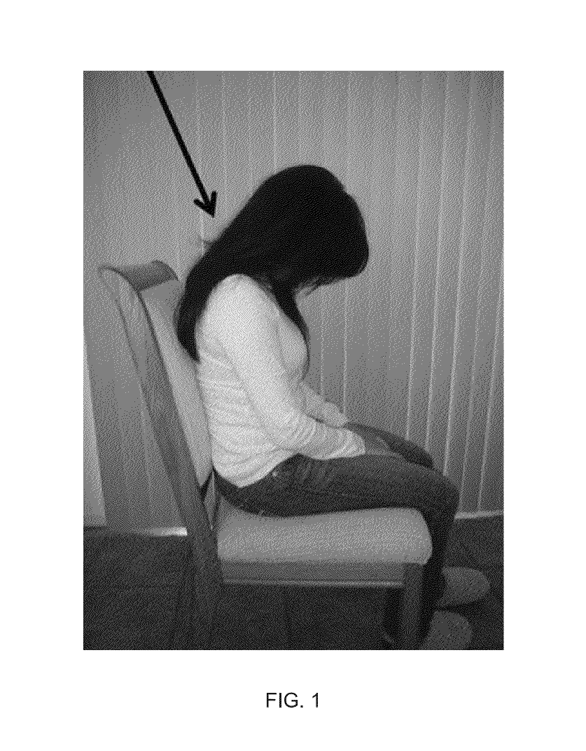

A wearer of the sleep mask who uses it during air travel, for example (or, more generally, in a seated position) expects to be able to sleep. The wearer's sleep, however, is well recognized to be rather uncomfortable because, during the sleep, muscles in the wearer's neck are relaxed and nothing prevents the head from falling down toward the chest, thus interrupting the sleep, as shown in FIG. 1. The related art is replete with descriptions of solutions to this problem, including so-called travel pillows and/or travel collars or rests that the wearer wraps around his neck to support his head during the sleep, and that, although light and often foldable, are bulky and require space in the traveler's luggage. The use of such neck-supporting solutions does not substitute the use of the sleep mask and, therefore, a typical air traveler has to use both the sleep mask (to block the light) and a neck-rest or pillow (to support his head from falling forward).

In accordance with embodiments of the present invention, methods and apparatus are disclosed configured to support a head of the sleeping user from at least falling forward, thereby keeping his head substantially upright while, at the same time, blocking the ambient light from reaching the user's eyes.

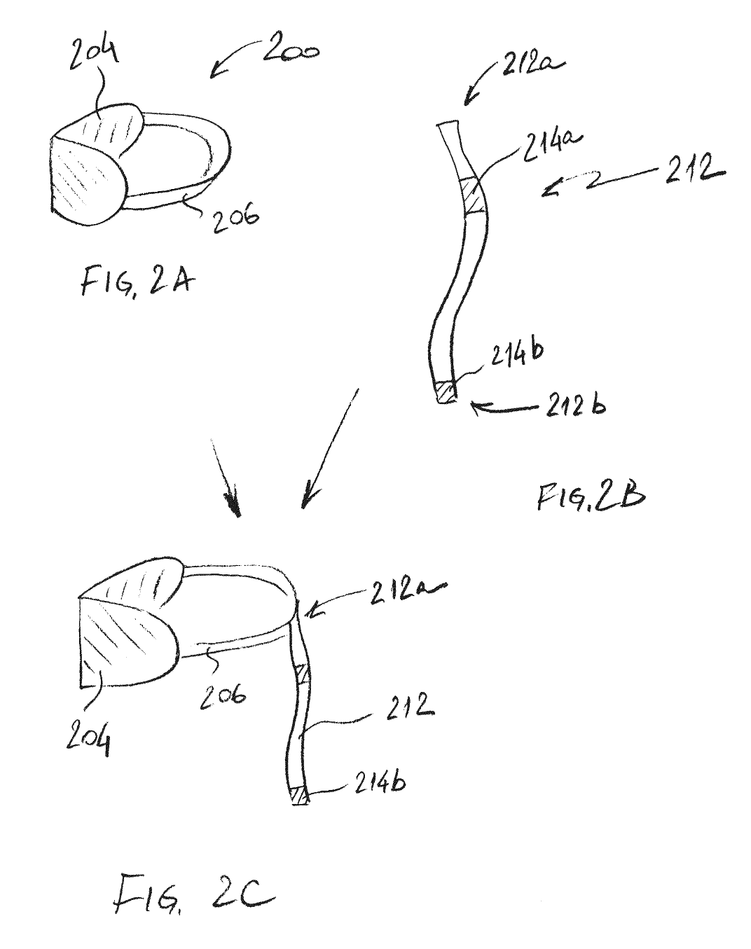

In reference to FIGS. 2A, 2B, and 2C, components of an embodiment of the invention are further described. As shown, a sleep mask 200 includes a blindfold portion 204 and a preferably substantially non-stretchable horizontal belt (such as flexible band or strap) 206 affixed at its both ends to the blindfold portion 204. The non-stretchability of the elements of the invention discussed below is specifically contrasted with the impressing deformation ability of the elements that include rubber bands or threads of various sorts, and is likened to that of a typical belt used as an element of clothing. In other words, the ratio between the final length of a substantially non-stretchable strap of the present invention, caused by stretching such a strap, and its initial length is comparable to that of a typical belt used as an element of clothing. The horizontal strap 206 is configured to fit around a head of the mask-wearer. It is appreciated that while a length of the strap 206 is substantially fixed, in practice the fit of the strap 206 around the user's head may be optionally made adjustable (as discussed below) to accommodate heads of different sizes. In one embodiment, the strap 206 is about ¾″ or 1″ wide and is made of Flat Pack Strap Webbing (John Howard Company, Chino, Calif.).

The sleep mask 200 is additionally equipped with a vertical strap 212 shown in FIG. 2B and having proximate and distal ends 212a, 212b and configured, in reference to the implementation of FIG. 2C, to be affixed (whether permanently or removably) to the horizontal strap 206 at its proximate end 212a such as to ensure that, when worn by the user, the vertical strap is substantially positioned along the back of the user. The vertical strap 212 is equipped with affixing means 214a, 214b such as, for example, Velcro-based fastener, a conventional buckle, a triglide buckle, a side-release buckle, or with other means configured to affix the vertical strap 212 to an element of clothing of the wearer of an embodiment of the invention. The vertical strap 212 is preferably made of the same material as the horizontal strap 206.

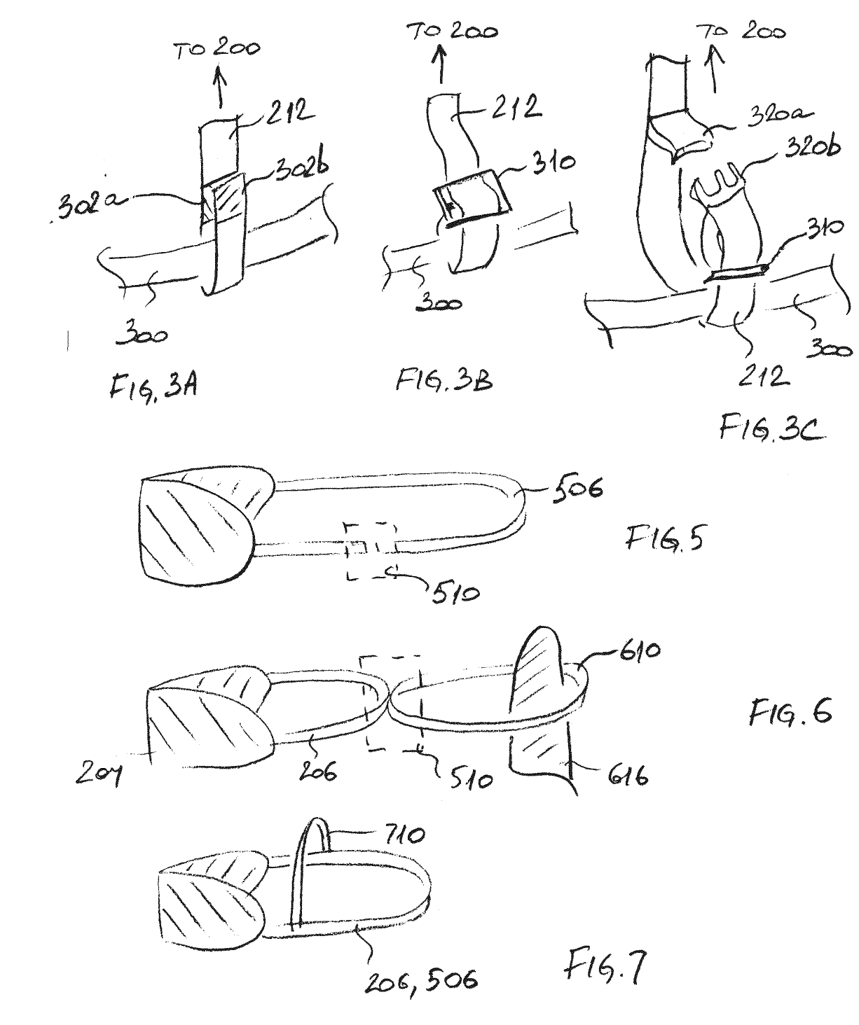

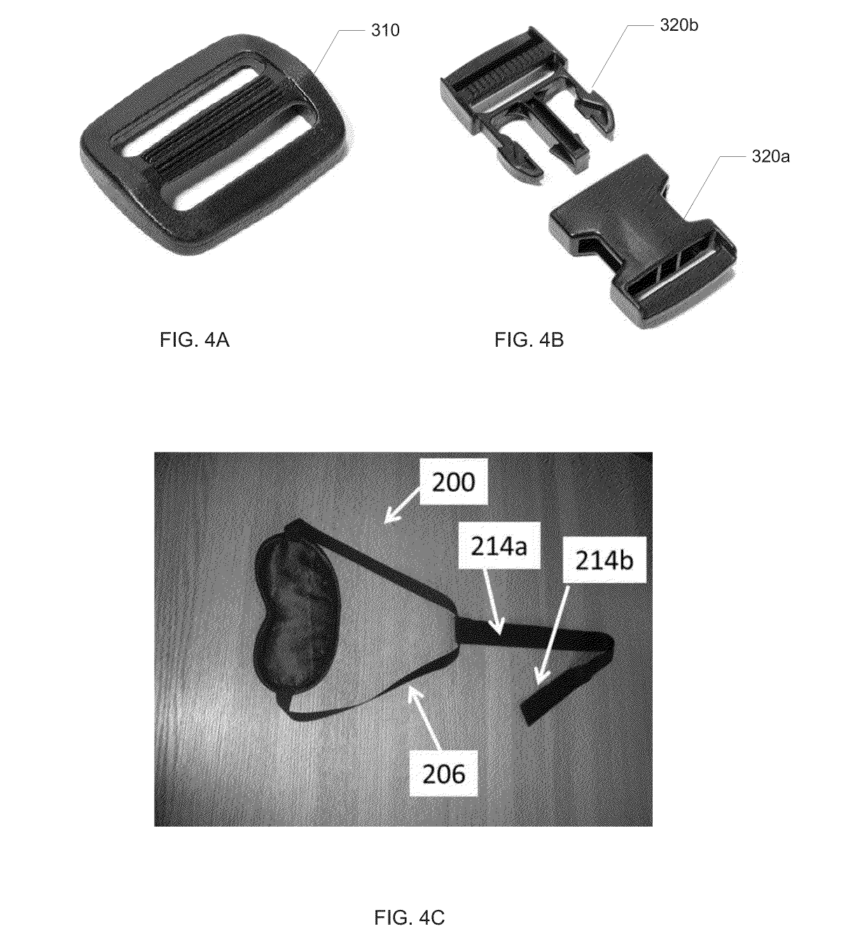

As shown in FIGS. 3A, 3B, and 3C, the vertical strap 212 is operably articulated, at its distal end 212b, with an element 300 of clothing of the wearer such as, for example, the wearer's belt or belt lashes at the back of the wearer's pants or skirt. The implementation of FIG. 3A makes use of the affixing means 214a, 214b that includes Velcro patches 302a, 302b. The embodiment of FIG. 3B employs a triglide buckle 310 such as that shown in FIG. 4. In comparison, the embodiment of FIG. 3C utilizes both the triglide buckle 310 and the side-release buckle 320a, 320b such as that shown in FIG. 4B. FIG. 4C provides a snap-shot of a prototype of the embodiment of the sleep mask system of FIG. 2C.

In a related embodiment, instead of using the wearer's belt of belt lashes, the vertical strap 212 may be alternatively wound around the wearer's torso or thigh, or attached or tied to an object located behind the sitting wearer such as, for example, the back of the seat. It is appreciated that at least one of the horizontal and vertical straps 206, 212 of the embodiments of FIGS. 2C, 4C is preferably configured to include a strap a fit of which to a portion of the user's body is adjustable and, in particular, tensionably adjustable. In one embodiment, for example, the fit of the vertical strap 212 defined as a linear extend of the strap 212 between its proximal end 212a and a point at which it reaches the article of clothing to which it is detachably affixed, is made adjustable by manipulating the affixing means 214a, 214b. To this end, and in further reference to FIG. 3A, an operationally optimized range of fit-adjustment is achieved by using a relatively long (for example, 9 to 13 inches or so) Velcro patch in place of the element 302a, while keeping the Velcro patch element 302b comparably short (for example, 2-4 inches or so) in order to facilitate opening and closing the Velcro fastener behind the person's back. In another example, an embodiment of FIG. 5 contains a horizontal strap 506 having a fit-adjusting mechanism 510 such as, for example, a Velcro-based or a buckle-based mechanism. A portion of the embodiment containing the vertical strap is not shown for simplicity of illustration.

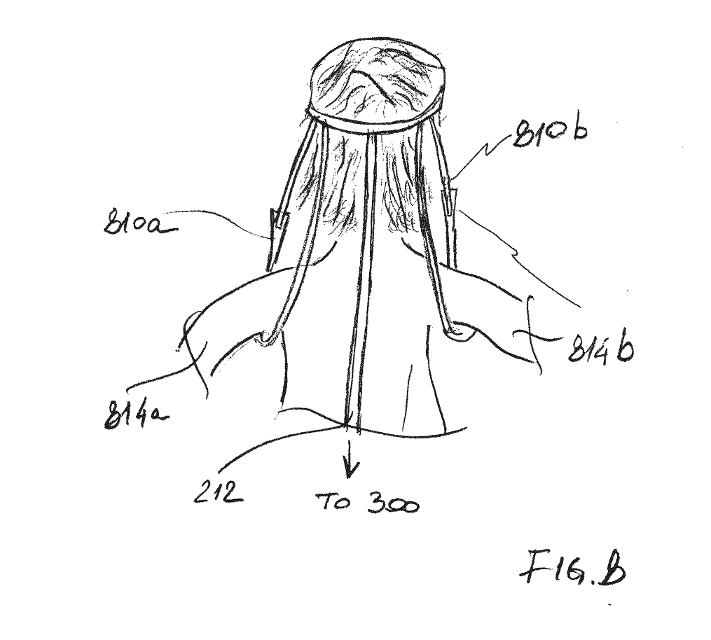

Referring now to FIG. 6, an alternative configuration of a sleep mask system is depicted that includes, in addition to the blindfolding portion 200 and the horizontal strap such as the strap 206 of FIG. 2A or the strap 506 of FIG. 5, an auxiliary horizontal strap 610 judiciously chosen to accommodate the attachment of the system, worn by the seated person (not shown), to an object 616 behind the person such a back of the seat, for example.

A further advantageous modification to the basic implementation of the portion 200 of the embodiment of FIG. 2C may include an adjustable-fit vertical-support strap 710 configured to be permanently or removably cooperated to the horizontal strap 206 or 506 such as to pass over the head of the wearer, when worn and to prevent the sleep mask system from sliding down the wearer's head. Such a related embodiment is schematically illustrated in FIG. 7.

In use, an extra advantage is gained if a sleep mask system is additionally equipped with side-stabilization components that are adapted to prevent the head of the wearer from falling sideways. An embodiment of the system containing such side-stabilization components 810a, 810b (as shown, side-stabilization straps) and worn by the user is shown from the rear of the user. The side-stabilization straps 810a, 810b are optionally length-adjustable, in a fashion similar to that discussed above, and are configured to be tensionably wrapped around the wearer's arms 814a, 814b. Alternatively, the side-stabilization straps 810a, 810b can be adapted to be removably attachable to an element of the wearer's clothing, for example to the sides of the belt worn by the person. Additionally or alternatively, the side-stabilization components 810a, 810b are configured to be removably attachable to an external object located on the sides of the seated person who wears the sleep mask system, for example, to the arm rests of the chair in which the person is sitting. The employ of the side-stabilization components 814a, 814b may be optimized by using these components with the embodiment of FIG. 506, which permits establishing a tight fit of the horizontal strap 506 around the wearer's head.

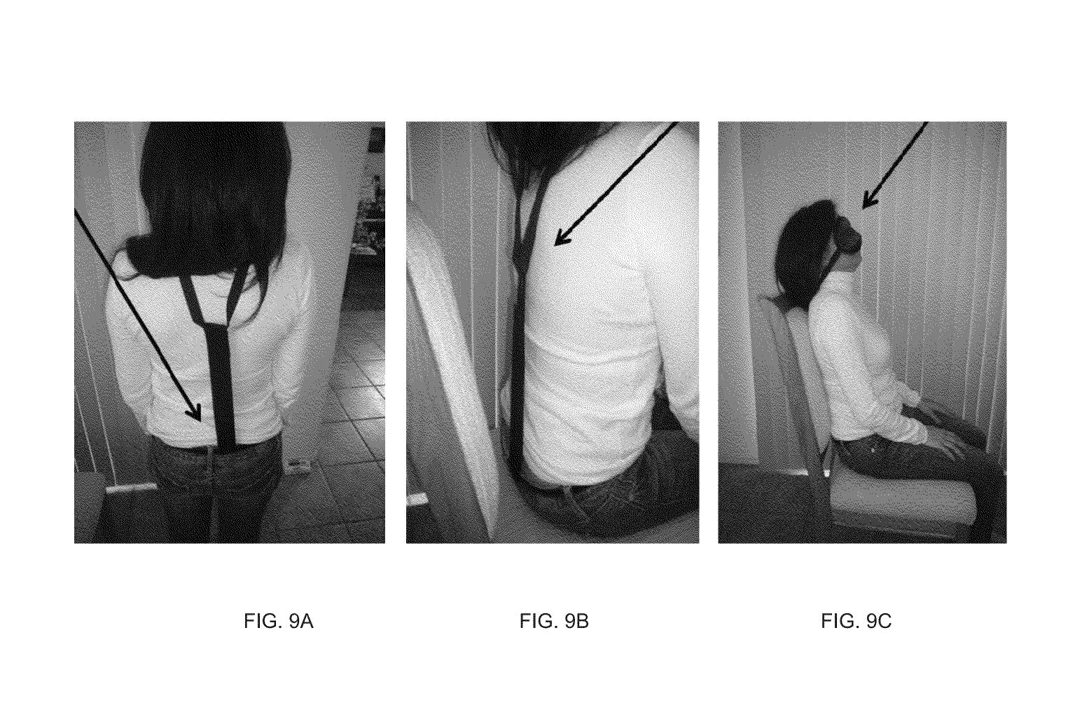

FIGS. 9A through 9C provide illustrations of the use of the sleep mask system of the invention and show the attachment of the harness element 212 to the wearer's belt and support of the head of the wearer in a substantially straight position while seated.

While specific examples of implementation of the sleep mask system has been discussed in reference to the particular drawings attached hereto, it is to be understood that modifications to, and variations of, the illustrated examples may be made without departing from the inventive concepts disclosed herein. Accordingly, the invention should not be viewed as being limited to the disclosed implementation(s). Examples of claims envisioned to claim the invention are provided by the appended tentative claims.

I claim:

1. A sleep mask system comprising:
a mask portion including
a blindfolding portion dimensioned to cover eyes of the user such as to block ambient light from reaching the eyes; and
a first substantially non-stretchable harness affixed to said blindfolding portion in circumscribing relationship with the head of the user such as to fasten the blindfolding portion over the eyes of the user;
and
a substantially non-stretchable suspending portion having a proximal end attached to the first harness at a point opposite the blindfolding portion and a distal end equipped with affixing means.

2. A system according to claim 1, further comprising a substantially non-stretchable side-stabilizing portion having an end attached to the first harness in proximity to the blindfolding portion.

3. A system according to claim 2, wherein said side-stabilizing portion is adapted to restrict a side movement of the head of the user wearing the mask portion.

4. A system according to claim 3, wherein said side-stabilizing portion has adjustable fit.

5. A system according to claim 1, wherein at least one of said first harness and suspending portion are adapted to have adjustable fit.

6. A system according to claim 1, wherein the affixing means is adapted to cooperate the suspending portion with an article of clothing of the user, who wears the mask portion around his head, such as to restrict a forward movement of the user's head.

7. A method for stabilizing a position of a head of a person, the method comprising:
providing a sleep mask that includes
a blindfolding portion configured to cover eyes of the person such as to block ambient light from reaching the eyes; and
a first substantially non-stretchable harness affixed to said blindfolding portion such as to circumscribe the head of the user and to fasten the blindfolding portion over the eyes of the user;
and
providing a substantially non-stretchable portion having a first end attached or attachable to said first harness and a second end removably affixable to a point distant from the head of the person such as to restrict a movement of the person's head in at least one of forward and sideway directions.

8. A method according to claim 7, wherein providing a substantially non-stretchable portion includes providing a substantially non-stretchable suspending portion having a proximal end attached to the first harness at a point opposite the blindfolding portion and a distal end equipped with affixing means configured to adjust a fit of said suspending portion between the proximal end and a point of affixing said suspending portion to the element of the person's clothing.

9. A method according to claim 7, wherein providing a substantially non-stretchable portion includes providing said non-stretchable portion having the second end that is removably affixable to an element of the person's clothing.

10. A method according to claim 7, wherein providing a substantially non-stretchable portion includes providing said non-stretchable portion having the second end that is adapted to be removably cooperated with an element of a seat when the person is seated in said seat.

11. A method according to claim 7, wherein providing a substantially non-stretchable portion includes providing a substantially non-stretchable side-stabilizing portion having an end attached to the first harness in proximity to the blindfolding portion and configured to have adjustable fit.

12. A sleep mask system comprising:
a mask portion including
a blindfolding portion which, when, cooperated with a user's head, covers eyes of the user such as to block ambient light from reaching the eyes; and
a first substantially non-stretchable harness affixed to said blindfolding portion such as to circumscribe the head of the user and to fasten the blindfolding portion over the eyes of the user, wherein said harness is adapted to have an adjustable fit to the user's head.

13. A system according to claim 12, further comprising a substantially non-stretchable suspending portion having a proximal end attached or attachable to the first harness at a first point distant from the blindfolding portion and a distal end equipped with means configured to connect said suspending portion to a second point distant from the blindfolding portion.
